(19) United States
(12) Plant Patent Application Publication
(10) Pub. No.: US 2010/0229268 P1
(43) Pub. Date: Sep. 9, 2010

(54) Pedro lopez late avocado (April)

(50) ;

(76) Inventor: Pedro M. Lopez, Miami, FL (US)

Correspondence Address: Pedro M. Lopez, 2530 SW 115 Avenue, Miami, FL, 33185, US

(21) Appl. No.: 12/317,235

(22) Filed: Mar. 9, 2009

(51) Int. Cl. A01H 5/00

(52) U.S. Cl. PLT/200

(57) ABSTRACT

A new variety of avocado is distinguished by its late maturing fruit. The tree blooms in March or early April and the blooms are of the B type, flowing. The fruit matures and can be picked from December to April of the following year. The fruit is obovate, fruit has a small flattened area toward the apex on one side, weighs between 16-21 ounces and averages 4 6/16 inches in diameter.

### BACKROUND OF THE INVENTION

For the past two years (1998-99), we have heard of an avocado with a very late maturity season. This avocado hangs on the tree until the end of March and the beginning of April. On the 16 of April 2000 we were able to see the tree. We also saw two fruits that had been kept in the refrigerator that had been picked the week of Apr. 9, 2000. This tree was discovered in 1976 in Pedro Lopez's previous residence at 4717 N.W. 1^{st} Street, Miami, Fla. 33135. Mr. Lopez moved this tree to his present residence at 2530 S.W. 115 Ave., Miami, Fla. 33165. This is the current location of the tree. The tree is a seedling and is large in both, width and height. The tree has type B flowers and there was an excellent fruit set. The tree is about 41 years old. There are only five (5) trees asexually propagated at Pedro Lopez' residence, 2530 SW 115 Avenue, Miami, Fla. 33165. No other asexual propagations have been made and will not be made until total patent approval is obtained. This cultivar is an open, pollinated seedling of unknown parents.

### BRIEF SUMMARY OF THE INVENTION

This cultivar differs from other known Florida varieties in that some fruits remain on the tree until March and April with a picking season from December to April. ‘April’ is currently listed in the United States Department of Agriculture (USDA) database, Biotechnology Section. Dr. Michael D. Sussman, molecular biologist, USDA, AMS, S&T, NSL, has declared that the USDA developed a set of micro-satellite markers that will allow differentiation of this cultivar from Monroe, Lula, Semil-34 and Semil-43, Choquette, Booth 8, Melendez, Hardee, Melendez and Simmonds, which are common late cultivars.

### LATIN NAME OF THE GENUS AND SPECIES

Persea americana Mill Family Lauraceae; ‘April’.

### DETAILED BOTANICAL DESCRIPTION

Below is the description of the tree and fruit characters made in 2000 with some additional information added during 2004.
Tree: Very large, very broad and very tall. The growth pattern is intermediate or spreading. It was originally a seedling. The tree was planted in 1963. The canopy is densely foliated. The tree was nearly destroyed in or about August 1992, by Hurricane Andrew that took place on or about Aug. 23-24, 1992. The tree was grafted on five occasions, including after Hurricane Andrew in 1992; after Hurricane Wilma in 2005; and after Hurricane Katrina in 2005. (FIG. 1). The type of grafting used was cleft grafting.
Height and spread of tree: The tree, located at 2530 SW 115 Avenue, Miami, Fla., is very large, very broad, and very tall that can reach about 35 feet high. The width of the canopy can reach approximately 30 to 60 feet, depending on the amount of pruning. The growth pattern is intermediate or spreading. The amount of growth of the tree is about 1½ feet to 2 feet per year. (FIG. 1).
Leaves: Fairly large, resembling Guatemalan type or hybrid. The alternate leaves are ovate and broad with a round base and pointed tips. Leaf blades are 6-10 inches long and 4-6 inches wide with an average length/width ratio of 1.6. The petioles are 1.3 to 1.7 inches long. The upper surface leaf color ranges from stock green to forest green or 003333 to 003300 (Coutsoukis, 2004) whereas the lower surface color ranges from odd green to seas spray (Hall Color Charts, 2004) or 336600 to 336633 (Coutsoukis, 2004). The leaf petiole and main veins are canary yellow (Hall Color Charts, 2004) or 669900 (Coutsoukis, 2004). The mean diameter of the base of the leaf petiole is 4.8 mm with a range of 4.3 mm to 5.3 mm. (FIG. 2.).
Flowers: Type B, stamens opening in the afternoon female and the next morning male. A few blooms left in Apr. 16, 2000 but mostly fruit set. The bloom season is probably late February to early April. The inflorescences are axillary borne near the base of the current season's growth but crowded at the ends of the branches so as often to appear apparently terminal, paniculate, many flowered, and on short or long stalks. The range of flowers per cluster is approximately 1,000 to 10,000. The rachis is tenete or compressed, yellowish-green (Crayola crayons 2004, code #52-0096-0-217), densely pubescent with numerous, oblong, lanceolate, yellowish-green, short tomentose, fugacious bracts. The small, bisexual, fragrant flower with a perfume-like fragrance, is ⅜″ to ½″ in diameter when expanded, are borne on short, terete, yellowish-green, densely tomentose pedicels. The corolla is lacking. The calyx is six-parted, yellowish-green or light yellow, usually densely, silky tomentose within and without, rarely subglabrous. The stamens number 12 in four whorls, the innermost series of three (3) being reduced to staminodes. The three innermost functional stamens are longer than the others with extrorse anthers and short-stalked citrine or orange-colored ovoid glands at the base of the filaments. The six outermost perfect stamens have introrse anthers and lack glands. The ovary is single-celled with a single style and dischaped stigma. (FIGS. 6 and 7).
Plant resistance/susceptibility: The leaves and fruit show a high degree of tolerance to diseases.
Fruit:
Shape: The fruit is obovate, fruit has a small flattened area toward the apex on one side (FIGS. 3 and 4).
Stem insertion and shape: The pedicel is inserted asymmetrically and is mostly conical, with some cylindrical (FIG. 3).
Size: Medium to medium large, total fruit wt 16-31 oz (average 25 oz), 3 14/16-4 6/16 inches in diameter, and 5 2/16-6 11/16 inches in length (FIG. 4) and length to width ratio of 1.2-1.3. Good size for Florida.
Peel: Peel wt 2.0-2.4 oz (average 2.2 oz). Average peel thickness ranges from 2.4 mm to 2.8 mm. The peel is stock green to forest green (green to dark green) (Hall Color Charts, 2004) or 336600 to 003300 (Coutsoukis, 2004), very slightly rough but smooth on the flattened area located toward the base (stylar end) of the fruit (FIG. 4). The fruit is shinny, with some very slight longitudinal ridges. A longitudinal lettuce colored (Hall Color Charts, 2004) or 66CC00 to 66CC33 (Coutsoukis, 2004) streak may be present on most fruit. The peel is slightly leathery and peeled easily from the pulp. No disease spots despite lack of fungicidal sprays were observed.
Pulp: On average the fruit is 84% pulp (FIGS. 4 and 5). The ratio of pulp wt to seed wt is 5.3. Pulp wt is 19-24 oz (average 21 oz). The pulp is generally medium in thickness however in some fruit it is a little less than medium. Pulp thickness ranges from 1 2/16-1 7/16 inches. The pulp is green adjacent to the peel and on average is 4/16 inch thick by 6/16 inch thick. Pulp color near the seed is yellow; near the peel Granny Smith Apple Green, Sheen Green or Forest Green (Crayola, 2004). It is deep yellow in color, surrounded in the periphery near the skin by a dark green thin ring of pulp about ¼ inches thick. There are no fibers in the pulp. Pulp consistency is smooth and buttery.
Seed: The seed is medium size (3.3-4.8 oz; mean 3.9 oz) and 2 3/16×2 8/16 inches (FIG. 4). The seed is tight (not loose) in the cavity. The average seed wt is 16% of total fruit wt. Seed coat color is brown to Sienna brown (Crayola, 2004). The seed coat does not adhere to pulp early in the season but may adhere later in the season. Cotyledon surface texture is very slightly rough to almost smooth.
Quality: Quality is excellent. The flesh was buttery, smooth, with an excellent nutty flavor. Pulp oil content is unknown.
Days from harvest to fruit softening: It took 5 to 8 days at 75° F. from picking to softening.
Production: Production for year 2001 was heavy, equivalent to 4 to 6 bushels (55 lbs per bushel) on a grove-size tree. The tree itself will probably bear 8 to 10 bushels, if not more. The tree bears every year but one year has a very heavy crop and the other a lighter crop. The estimated crop in a light year was about 1.5 bushels. The tree receives minimal care. No minor element sprays, iron drenches and only a small amount of dry fertilizer were being applied regularly.
Comments: Because of the outstanding and unique horticultural characters, especially its very late season, this cultivar should be planted in a grove setting for further evaluation. This cultivar should command excellent prices due to its late harvest season.

### REFERENCES

1. Hall Color Charts. [web page] 20 Aug. 2002; http//:silvaire.home. att.net/colors/chart.htm. [Accessed 2 Dec. 2004].
2. Coutsoukis, P. [web page]; http://www.immigration-usa.com/html_colors.html [hosted by http://www.theodora.com/ita.html, Accessed 2 Dec. 2004].
3. Crayola Crayons. 2004. 96 Crayola Crayons, Code #52-0096-0-217. Binney and Smith, Easton, PA.

### BRIEF DESCRIPTION OF THE DRAWINGS

FIG. 1 shows the height and width and growth pattern of the avocado tree.

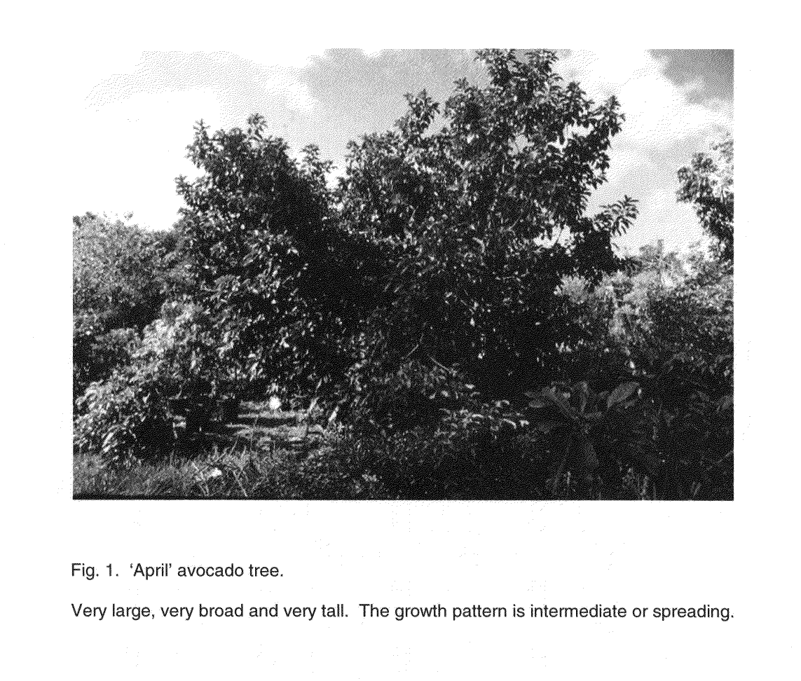

FIG. 2 shows the leaf size and color.

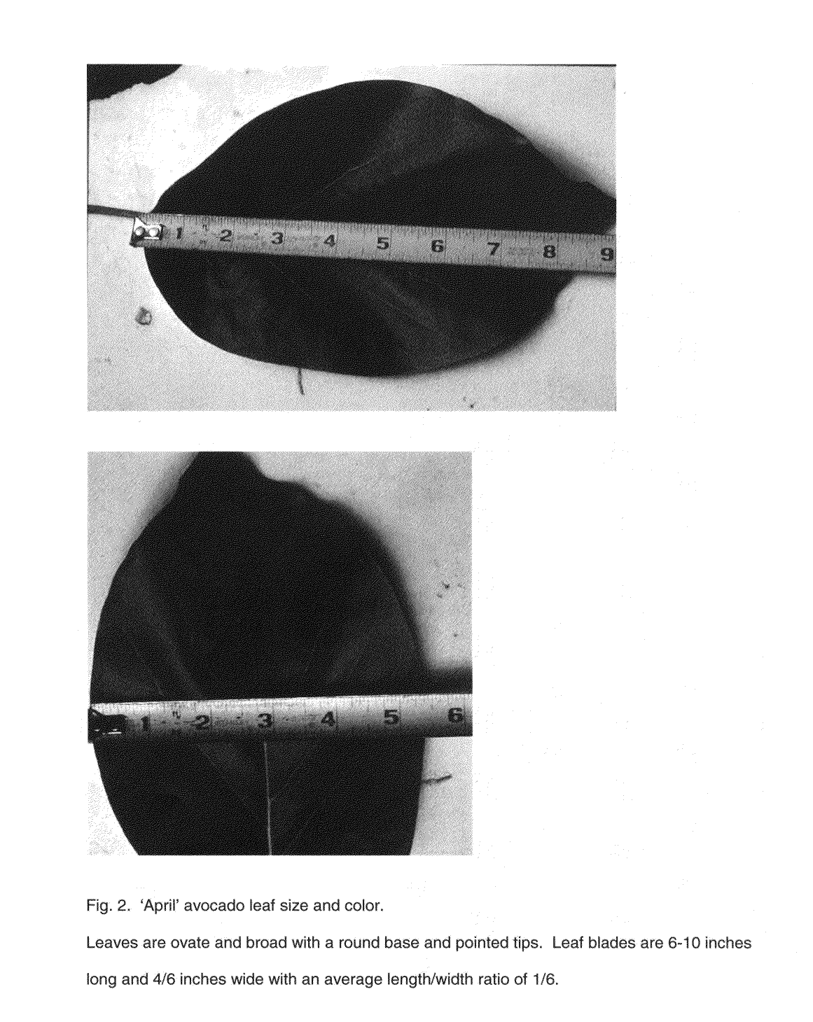

FIG. 3 shows the fruit size and shape.

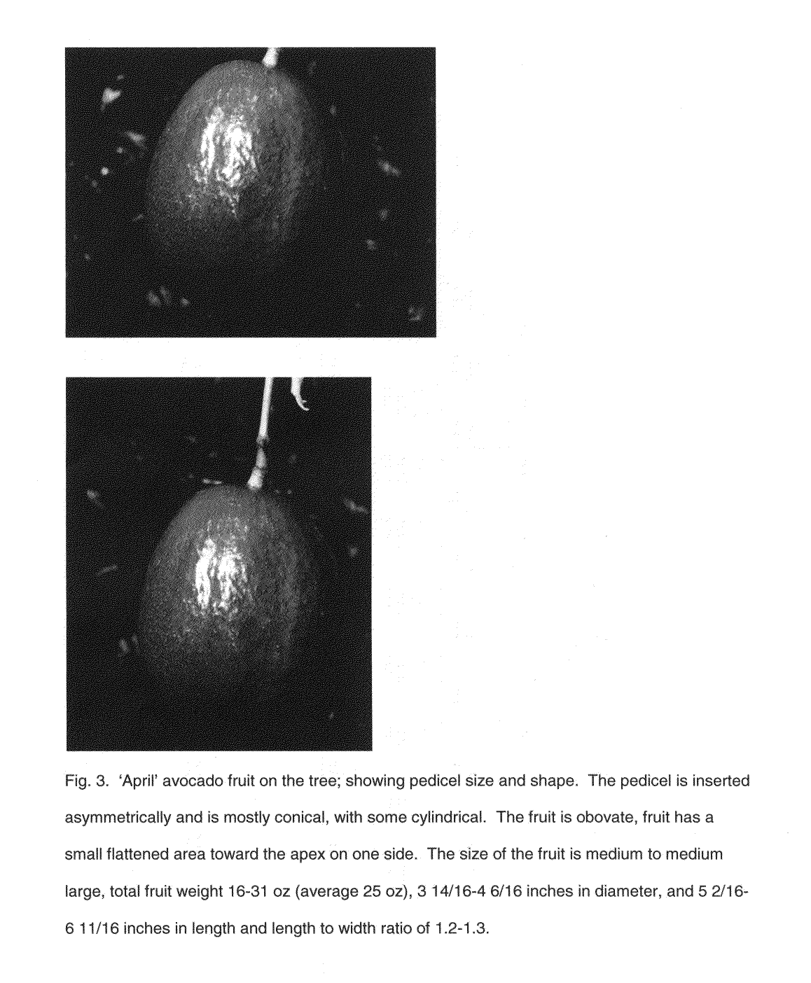

FIG. 4 shows the avocado cut and non-cut fruit.

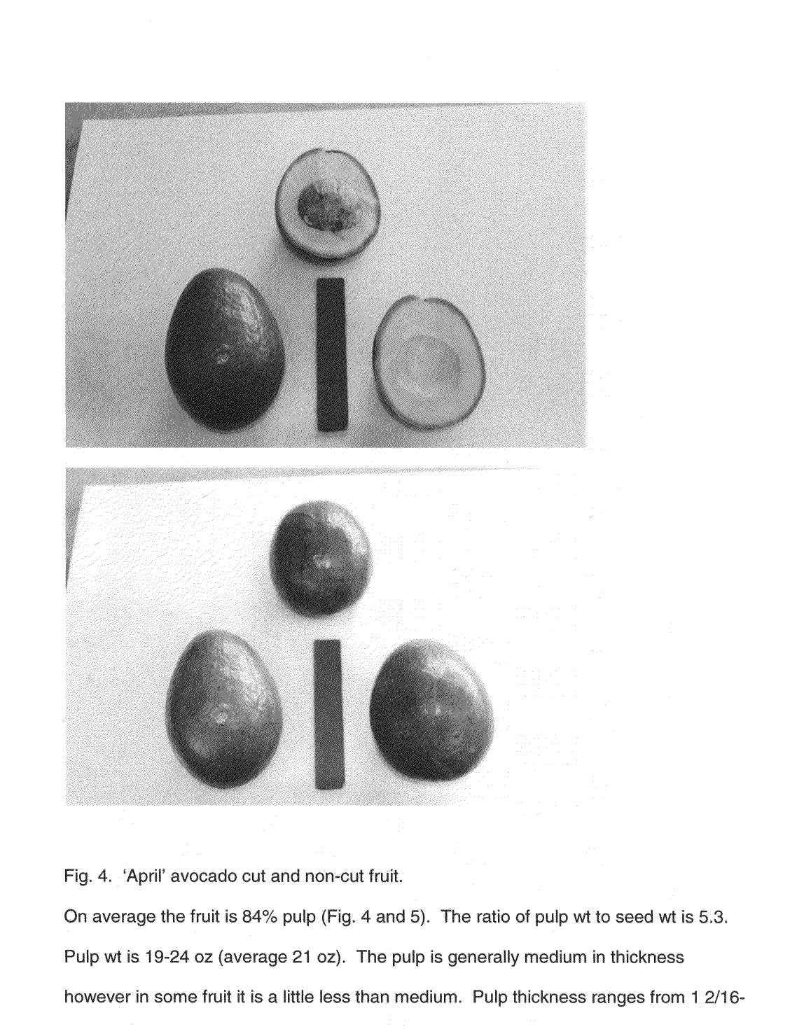

FIG. 5 shows the avocado cut fruit.

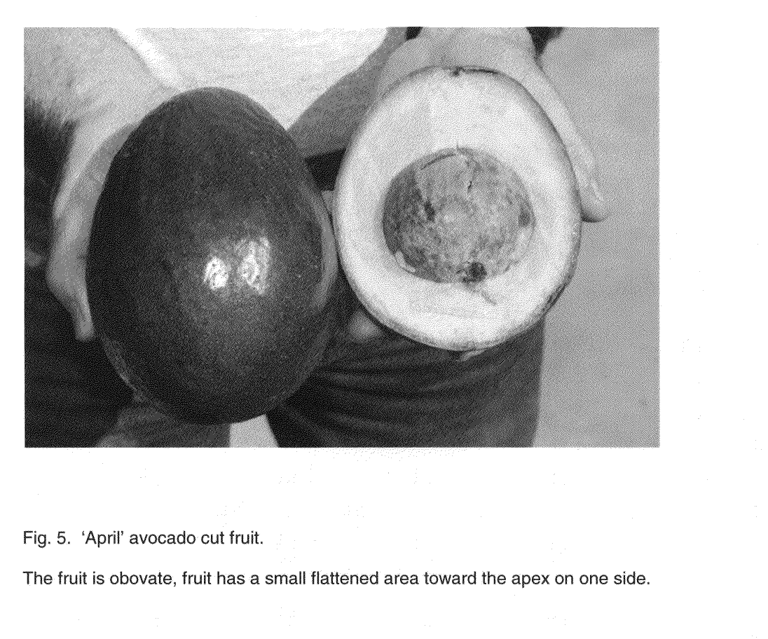

FIG. 6 shows the flowering stage female (Mid-Afternoon)

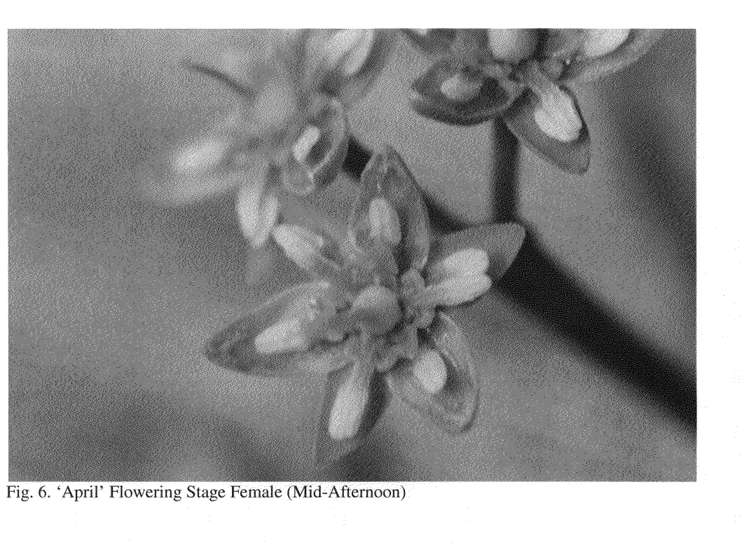

FIG. 7 shows the flowering stage male (Mid-Morning)

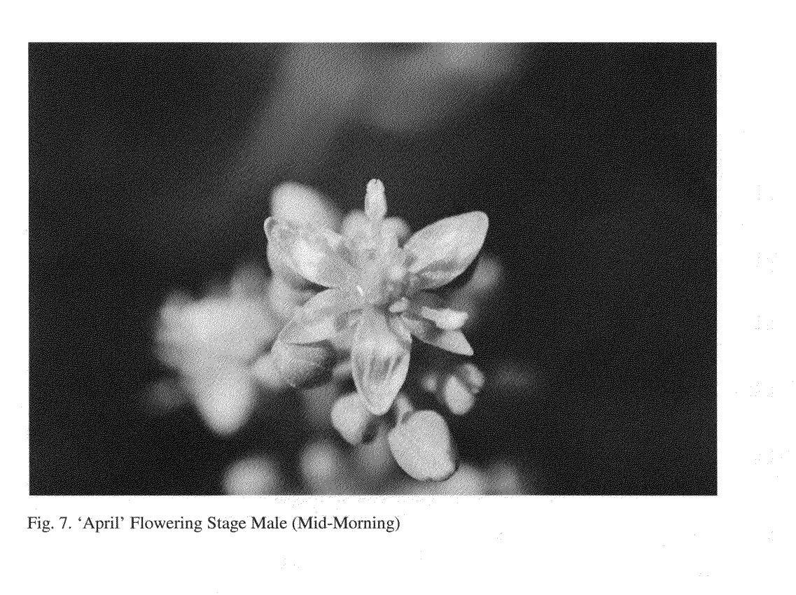

FIG. 8 shows the complete panicles.

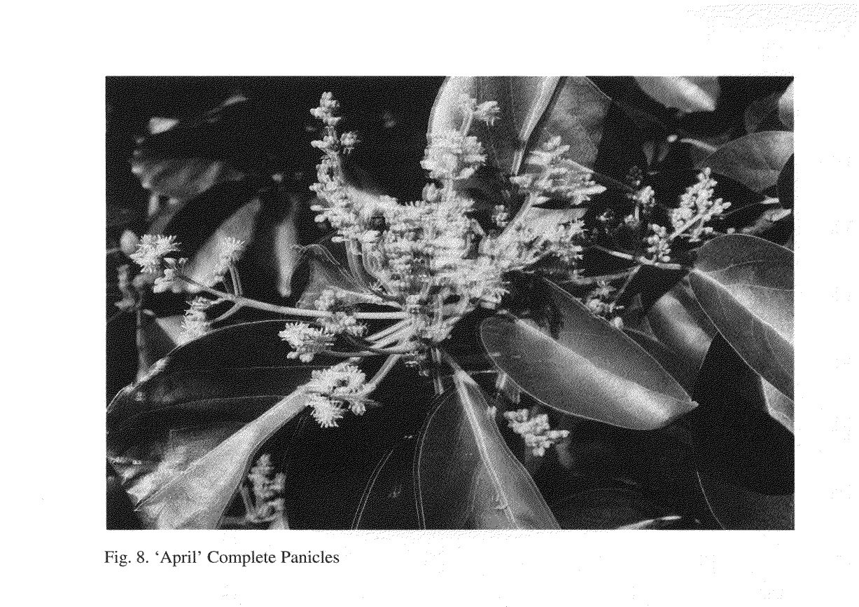

FIG. 9 DNA

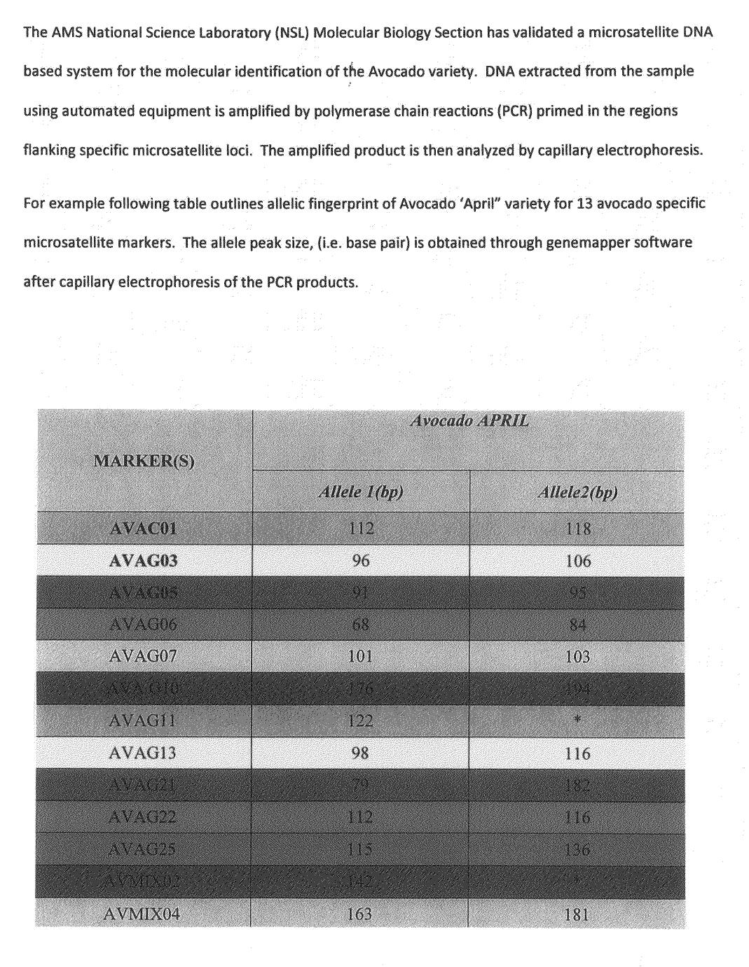

I claim:

1. A new and distinct variety of avocado tree substantially as described and illustrated and characterized as to novelty by its maturing very late in the season.
